(19) United States
(12) Patent Application Publication
(10) Pub. No.: US 2022/0028533 A1
(43) Pub. Date: Jan. 27, 2022

(54) METHOD AND SYSTEM FOR IDENTIFYING INFECTION HOTSPOTS IN HOSPITALS

(71) Applicant: KONINKLIJKE PHILIPS N.V., EINDHOVEN (NL)

(72) Inventors: Chaitanya KULKARNI, Bangalore (IN); Mohammad Shahed SOROWER, Natick, MA (US); Bryan CONROY, Garden City South, NY (US); Claire Yunzhu ZHAO, Cambridge, MA (US); David Paul NOREN, Sharon, MA (US); Kailash SWAMINATHAN, Chennai (IN); Ting FENG, Cambridge, MA (US); Kristen TGAVALEKOS, Cambridge, MA (US); Daniel Craig MCFARLANE, Reading, MA (US); Erina GHOSH, Boston, MA (US); Vinod KUMAR, Bangalore (IN); Vikram SHIVANNA, Bangalore (IN); Shraddha BARODIA, Bangalore (IN); Emma Holdrich SCHWAGER, Cambridge, MA (US); Prasad RAGHOTHAM VENKAT, Bangalore (IN)

(21) Appl. No.: 17/267,615

(22) PCT Filed: Apr. 10, 2020
(86) PCT No.: PCT/EP2020/060303

§ 371 Date: Feb. 10, 2021

Related U.S. Application Data
(60) Provisional application No. 62/833,152, filed on Apr. 12, 2019.

(51) Int. Cl. G16H 40/20; G16H 50/30; G16H 50/80; G06N 7/00

(52) U.S. Cl. CPC G16H 40/20; G06N 7/005; G16H 50/80; G16H 50/30

(57) ABSTRACT

A method for processing medical information includes identifying a first patient in a first state, identifying a second patient in a second state, calculating a first risk score for the first patient, calculating a first risk score for the second patient, and determining a risk prone area in a medical facility based on the first risk score for the first patient and the first risk score for the second patient. The first state is an infected state and the second state is different from the first state. The first risk score of the first patient provides an indication of a severity of the infected state of the first patient, and the first risk score of the second patient provides an indication of the second patient being infected by the first patient.

### STATEMENT OF GOVERNMENT SPONSORED SUPPORT

This invention was made with Government support under Agreement No. W15QKN-17-9-0008 awarded by USACC-NJ. The Government has certain rights in the invention.

### TECHNICAL FIELD

This disclosure relates generally to processing information, and more specifically, but not exclusively, to identifying health conditions in a medical facility.

### BACKGROUND

Persons at hospitals and other medical facilities are routinely exposed to pathogens that may cause disease or infection. Exposure to pathogens may occur through close contact with people who are either infected or carriers of pathogens that cause infection. Exposure to pathogens may also occur through physical contact with objects (e.g., tables, knobs, handles, etc.) which have been previously touched by infected people. According to the Centers for Disease Control and Prevention (CDC), on any given day, about one in 25 hospital patients develops a hospital-acquired-infection (HAI) through these or other means.

Some members of the population tend to be at higher risk than others. For example, very young people and very old people are at increased risk to HAIs because of their underdeveloped or weakened immune systems. Others have certain types of medical conditions (e.g., sepsis, cancer, etc.) or are undergoing medical treatments (e.g,. chemotherapy, radiation patients, steroids, major surgery, etc.) that present an increased risk to HAIs.

Presently, there are no reliable ways for determining areas in medical facilities that are harboring pathogens or which otherwise may be labeled as “hot spots” for exposure to disease and infection. Consequently, people continuing getting sick in the very places they go to seek healing.

### SUMMARY

A brief summary of various example embodiments is presented below. Some simplifications and omissions may be made in the following summary, which is intended to highlight and introduce some aspects of the various example embodiments, but not to limit the scope of the invention. Detailed descriptions of example embodiments adequate to allow those of ordinary skill in the art to make and use the inventive concepts will follow in later sections.

In accordance with one embodiment, a method for processing medical information includes identifying a first patient in a first state, identifying a second patient in a second state, calculating a first risk score for the first patient, calculating a first risk score for the second patient, and determining a risk prone area in a medical facility based on the first risk score for the first patient and the first risk score for the second patient. The first state is an infected state and the second state is different from the first state. The first risk score of the first patient provides an indication of a severity of the infected state of the first patient, and the first risk score of the second patient provides an indication of the second patient being infected by the first patient.

The first risk score of the second patient may be calculated based on the first risk score of the first patient and a gamma value, the gamma value corresponding to a probability that the second patient will be infected by the first patient. The gamma value may be calculated based on a location of the second patient relative to a location of the first patient in the medical facility. The gamma value may be calculated based on a type of separator between the first patient and the second patient. The gamma value may be calculated based on one or more procedures or protocols at the medical facility.

The method may include determining a first location in the medical facility to move the second patient relative to a location of the first patient, calculating a second risk score for the second patient at the first location, and selecting the first location if the second risk score for the second patient indicates a lower probability that the second patient will be infected by the first patient than the first risk score of the first patient. The method may include identifying one or more actions to reduce the first risk score of the second patient.

The method may include identifying a third patient in the first state, calculating a risk score for the third patient, calculating a second risk score for the second patient based on the risk score of the third patient, and calculating a third risk score for the second patient based on the first risk score of the second patient and the third risk score for the second patient. The first patient and the third patient may have different infections or are in different stages of a same infection. The risk score of the third patient may be different from the first risk score of the first patient. The method may include determining a plurality of locations in the medical facility to move the second patient relative to locations of the first and third patients; and selecting one of the plurality of locations using a Markov chain that generates different probabilities corresponding to the plurality of locations.

In accordance with another embodiment, a system for processing medical information includes a storage area to store an algorithm and a processor to implement the algorithm to calculate a first risk score for a first patient in a first state, calculate a first risk score for a second patient in a second state, and determine a risk prone area in a medical facility based on the first risk score for the first patient and the first risk score for the second patient, The first state is an infected state and the second state is different from the first state. The first risk score of the first patient provides an indication of a severity of the infected state of the first patient, and the first risk score of the second patient provides an indication of the second patient being infected by the first patient.

The processor may calculate the first risk score of the second patient based on the first risk score of the first patient and a gamma value, the gamma value corresponding to a probability that the second patient will be infected by the first patient. The processor may calculate the gamma value based on a location of the second patient relative to a location of the first patient in the medical facility. The processor may calculate the gamma value based on a type of separator between the first patient and the second patient. The processor may calculate the gamma value based on one or more procedures or protocols in place at the medical facility.

The processor may determine a first location in the medical facility to move the second patient relative to a location of the first patient, calculate a second risk score for the second patient at the first location, and select the first location if the second risk score for the second patient indicates a lower probability that the second patient will be infected by the first patient than the first risk score of the first patient. The processor may identify one or more actions to reduce the first risk score of the second patient. The processor may identify a third patient in the first state, calculate a risk score for the third patient, calculate a second risk score for the second patient based on the risk score of the third patient, and calculate a third risk score for the second patient based on the first risk score of the second patient and the third risk score for the second patient. The processor may determine a plurality of locations in the medical facility to move the second patient relative to locations of the first and third patients, and select one of the plurality of locations using a Markov chain that generates different probabilities corresponding to the plurality of locations.

In accordance with another embodiment, a non-transitory, machine-readable medium stores instructions for controlling a processor to calculate a first risk score for a first patient in a first state, calculate a first risk score for a second patient in a second state, and determine a risk prone area in a medical facility based on the first risk score for the first patient and the first risk score for the second patient. The first state is an infected state and the second state is different from the first state. The first risk score of the first patient provides an indication of a severity of the infected state of the first patient, and the first risk score of the second patient provides an indication of the second patient being infected by the first patient. The instructions may also control the processor to calculate the first risk score of the second patient based on the first risk score of the first patient and a gamma value, the gamma value corresponding to a probability that the second patient will be infected by the first patient.

### BRIEF DESCRIPTION OF THE DRAWINGS

The accompanying figures, where like reference numerals refer to identical or functionally similar elements throughout the separate views, together with the detailed description below, are incorporated in and form part of the specification, and serve to further illustrate example embodiments of concepts found in the claims and explain various principles and advantages of those embodiments.

These and other more detailed and specific features are more fully disclosed in the following specification, reference being had to the accompanying drawings, in which:

FIG. 1 illustrates an embodiment of a method for managing medical information;

FIGS. 2A-2D illustrates examples of various scenarios to be managed by the method;

FIG. 2E illustrates an example of an algorithm to compute risk scores;

FIG. 3A and 3B illustrate embodiments for generating risk scores;

FIG. 4 illustrates another embodiment of a method for managing medical information;

FIG. 5 illustrates another embodiment for generating a risk score; and

FIG. 6 illustrates an embodiment of a system for managing medical information.

### DETAILED DESCRIPTION

It should be understood that the figures are merely schematic and are not drawn to scale. It should also be understood that the same reference numerals are used throughout the figures to indicate the same or similar parts.

The descriptions and drawings illustrate the principles of various example embodiments. It will thus be appreciated that those skilled in the art will be able to devise various arrangements that, although not explicitly described or shown herein, embody the principles of the invention and are included within its scope. Furthermore, all examples recited herein are principally intended expressly to be for pedagogical purposes to aid the reader in understanding the principles of the invention and the concepts contributed by the inventor(s) to furthering the art and are to be construed as being without limitation to such specifically recited examples and conditions. Additionally, the term, “or,” as used herein, refers to a non-exclusive or (i.e., and/or), unless otherwise indicated (e.g., “or else” or “or in the alternative”). Also, the various example embodiments described herein are not necessarily mutually exclusive, as some example embodiments can be combined with one or more other example embodiments to form new example embodiments. Descriptors such as “first,” “second,” “third,” etc., are not meant to limit the order of elements discussed, are used to distinguish one element from the next, and are generally interchangeable. Values such as maximum or minimum may be predetermined and set to different values based on the application.

Example embodiments describe a system and method for identifying areas in a medical facility that are considered to be “hot spots” for exposure to pathogens or which otherwise may lead to disease or infection. This is accomplished, for example, by identifying areas with patients who may have an increased vulnerability to developing infection and/or by identifying infected patients who pose a risk of transmitting pathogens to others. By identifying these areas or patients, the spread of HAIs may be curtailed or even prevented.

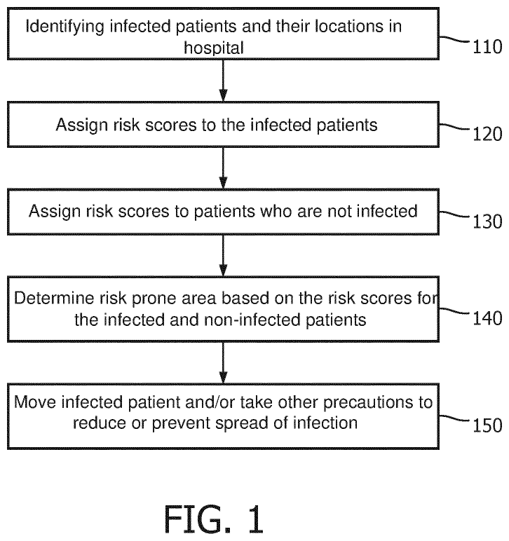

FIG. 1 illustrates an embodiment of a method for managing the spread of infection and disease (e.g., HAIs) in a medical facility. The medical facility may include a hospital, doctor office, surgery center, health clinic, or any other area where infected or vulnerable persons may be located. For convenience, the medical facility will be discussed as a hospital in the following description.

In operation 110, the method includes identifying patients in the hospital who are infected with one or more predetermined diseases or pathogens. This operation may be performed for all patients in the hospital or patients in one or more predesignated areas, care units, floors, or other zones in the hospital. The infected patients may be identified by name (or other identifier) and/or location in the hospital. In one embodiment, the patient may be identified by location in the hospital, e.g,. patient bed, patient room, area of patient treatment, and/or other location where patients may reside or otherwise be cared for.

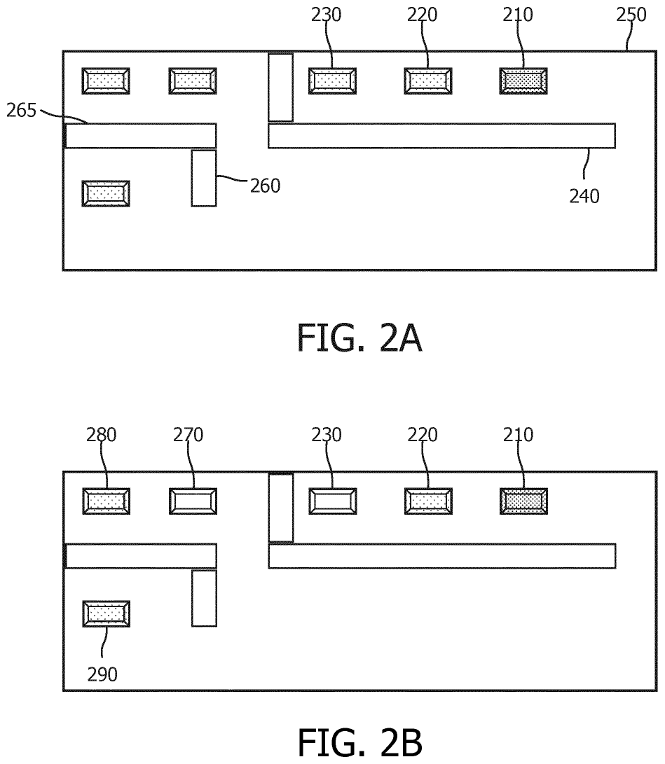

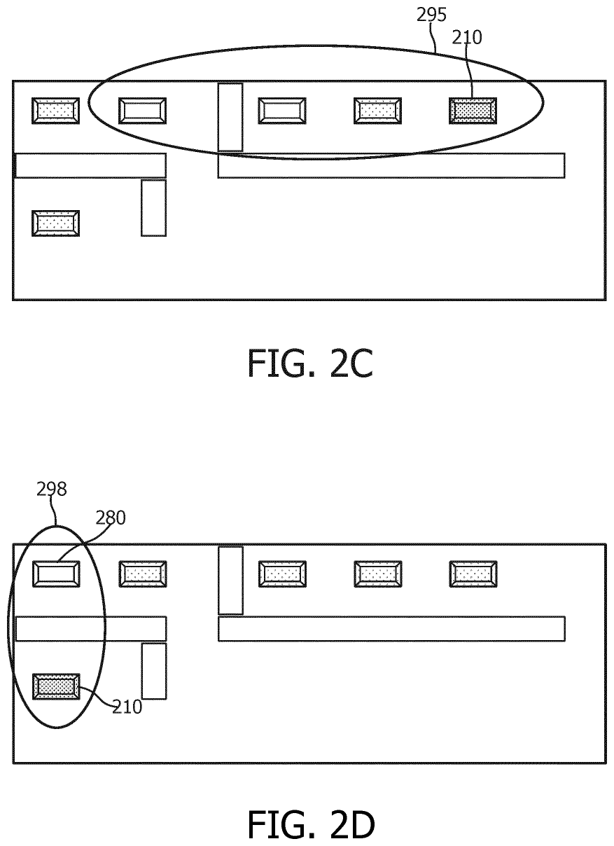

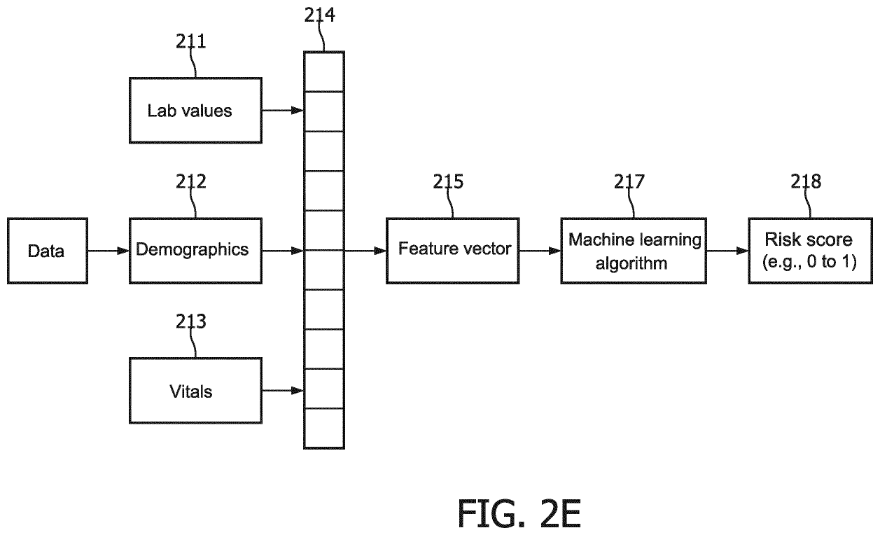

An example is illustrated in FIG. 2A, where the location of an infected patient is identified based on the bed 210 he is assigned to. In this case, the bed of the infected patient is in a section 240 of a ward 250 shared with other patients in beds 220 and 230. In the illustrated example, the ward may include other sections 250 and 260 that include beds assigned to patients who are also not infected. Partitioning of the sections may be accomplished, for example, by a screen or another type of divider. All patients in the ward may be considered vulnerable to infection (e.g., because of the inability of the dividers to quarantine the infected patient from the non-infected patients, because of contact from nurses who are handling the patients in the ward, etc.)

Some types of infections may be more contagious than others. In one embodiment, infections that are considered to be of greatest concern may be identified in operation 110. In other embodiments, patients may be categorized by type of infection. The patients may be identified, for example, based on stored information providing a list of the types of infections that are of concern, as determined by healthcare professionals beforehand. Examples of HAIs include but are not limited to the influenza, hepatitis, HIV, meningitis, tuberculosis, and Cholera.

In operation 120, an infection risk score Rᵢ is assigned to each infected patient. The risk score for each infected patient (or, synonymously, each bed) may be determined in various ways. In one embodiment, the risk score Rᵢ for each infected patient may be based on factors including the length of time the patient has been infected, the severity of the infection, and the course of antibiotics (or other medicine or treatment) the patient is being given. In one embodiment, weights may be assigned to these factors to indicate, for example, different levels of importance or severity of infection in calculating the risk score. The risk scores Rᵢ may have higher values for patients who have a more severe infection or who present a greater risk of infection to others, e.g., who are considered to have infections considered to be more highly contagious than other types of infections. The risk scores may be lower for less severe and/or less contagious infections.

In accordance with one embodiment illustrated in FIG. 2E, the risk score for a patient may be determined by calculating a feature vector 215 based on lab values 211, demographics data 212, and/or vitals features 213 recorded for the patient. The lab values 211 may include, for example, clinical laboratory test scores, e.g,. WBC, Creatinine, and Bicarbonate. These tests may be administered, for example, once a day or intermittently when, for example, the patient is in a hospital. The demographics data 212 may include, for example, age, height, weight, etc. This information may be drawn from various sources including medical records stored in the medical facility or obtained from one or more remote sources. The vitals features 213 may include, for example, temperature, blood pressure, heart rate, etc. Once this information has been collected, the feature vector 215 may be calculated, for example, using one or more known algorithms 214.

After the feature vector 15 has been calculated, a machine learning algorithm 217 may be used to generate a risk score 218 for the patient. In one implementation, the machine learning alrogithm 217 may include a classification algorithm which generates a probability score in a predetermined range, e.g., between 0 and 1, based on the feature vector. In one embodiment, values closer to 0 indicate a low risk that the patient is infected and values closer to 1 indicate a higher risk that the patient is infected.

In operation 130, risk scores Rₙᵢ are assigned to patients in the hospital who are not considered to be infected. In one embodiment, risk scores Rₙᵢ are assigned to every patient (or bed) in the hospital. In other embodiments, risk scores may be assigned to patients who are only in certain zones or areas of the hospital and/or who are in a certain range or proximity to infected patients.

The risk scores Rₙᵢ may be calculated by an algorithm that may take the following factors into consideration: proximity of the bed of an infected patient to the beds of patients who are not infected, the type of separator between the bed of the infected patient and the beds of the not-infected patient (e.g., wall, curtain, hallway, no separator such that the beds of the infected and not-infected patients are in same room), identified pathogen of infection (e.g., highly contagious, moderately contagious, not very contagious), type of exposure (e.g,. patients sharing the same nurse, cleaners, healthcare provders, or other members or employees of the hospital, patients who have undergone the same procedures or treatments, patients having comorbidity, etc.), as well as other factors that may influence separation between the bed of the infected patient and the beds of not-infected patients.

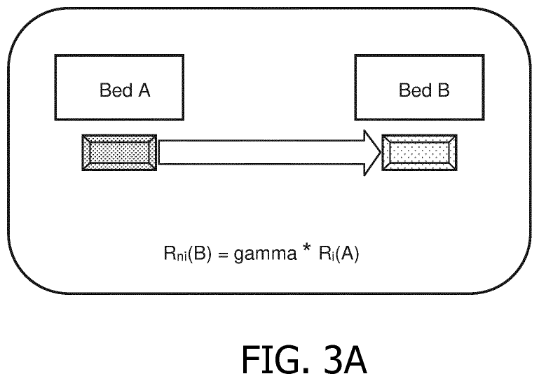

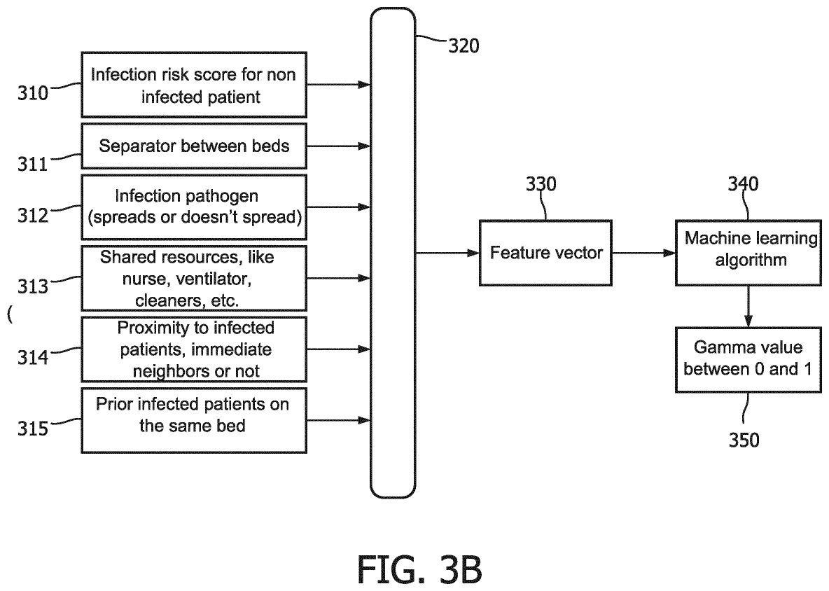

An example of an algorithm that may take these and/or other factors into consideration for purposes of calculating the risk scores of patients who are not infected may be based on Equation 1, where Rₙᵢ indicates the risk score assigned to a patient who is not infected but is in the vicinity of patient who is infected and Rᵢ corresponds to the risk score calculated for the infected patient in operation 120. FIG. 3A illustrates this equation in relation to infected patient A and not-infected, neighboring patient B.

Rₙᵢ=Gamma*Rᵢ (1)

In equation 1, gamma may correspond to a weighted value, for example, between 0 and 1. This weighted value may be computed based on the relative location, separator type, protocols or precedures in place, and/or any of the other aforementioned factors, which themselves may be assigned weights according to predetermined degrees of importance. Because the value of gamma be between 0 and 1, the risk scores assigned to patients that have some risk of infection will be a value less than (e.g., discounted from) the value of the risk score of the infected patient. Patients with no risk of infection will have a gamma value of 0, and the infected patient will have a gamma value of 1.

FIG. 3B illustrates an example of an algorithm for calculating a risk score for a non-infected patient. The algorithm includes calculating the gamma value in Equation 1, first, by generating a feature vector 330 based on, for example, the separator between beds 311, infection pathogen 312, shared sources 313, proximity to infected patients 314, and prior infected patients on the same bed 315. A value may be assigned to or calculated for each of these features, e.g., based on recorded or historical data, statistical data, and/or by one or more predetermined algorithms.

For example, the value for the separator between beds may be a value based on categories including are same room-immediate neighbor, same room-non-neighbor, different room-immediate neighbor, same floor, etc. The value for an infection pathogen may be based on categories including spreads via air, spreads via water, spreads via bodily fluid or contact, etc. The value for shared resources may be based on categories including same ventilator, same bathroom, same nurse, etc. In one embodiment, use of a shared nurse may have its own categorical values, for example, based on the type of nurse being shared. The value for prior patient condition on the same bed may be based on whether the prior patient was infected or not infected.

Once all of these values are determined, an algorithm 320 may be used to generate the feature vector 330. The algorithm may be, for example, a classification algorithm that generates the feature vector based on the input values. The feature vector 330 may then be input into a machine learning algorithm 340 which generates the gamma value of equation 1. In one embodiment, the machine learning algorithm may generate the risk score for the non-infected patient based on the product of the gamma value and the risk score 310 generated for the infected patient. The risk score 350 for the non-infected patient provides an indication of the susceptibility of this patient to become infected by the infected patient.

A possible scenario involving the assignment of risk scores Rₙᵢ relative to an infected patient is illustrated in FIG. 2B. Here, beds 220 and 230 are given progressively lower risk scores with increasing distance from the bed 210 of the infected patient, in bed 210. Another bed 270 at a more distant location in the same ward is given a lower risk score, and the remaining two beds 280 and 290 in the ward are assigned risk scores indicating risk of infection from the infected patient.

In one embodiment, a graphical respresentation as shown in FIG. 2B may be generated and output on a display with color-coded and/or other indicia indicating the relative relationship of the scores. For example, the bed of the infected patient may be red, the beds of the patients with no risk of being infected may be green, and the beds of patients with varying degrees of infection risk may have different corresponding shading of the same color or different colors.

The risk scores (e.g., the gamma values) for the patients who are not infected may be different, for example, based on the placement of certain separators and/or other factors, e.g., types of medical procedures or precautions taken or preventative measures that are in place. For example, the risk score of a patient closer to an infected patient may be lower than the risk score for a patient located farther away from the infected patient if a separator which provides improved protection against the transfer of pathogens is placed between the closer, not-infected patient and the infected patient.

In operation 140, at least one risk prone area RPA is determined based on the risk scores Rᵢ and Rₙᵢ for the infected and not-infected patients. The risk prone area RPA may be determined, for example, by extrapolating the risk scores Rᵢ and Rₙᵢ onto a floor level or other area. An example of an RPA determined for the case in FIG. 2B is indicated by area 295 in FIG. 2C. In this example, the risk prone area 295 is determined to include all patients 220, 230, and 270 having a non-zero risk score Rₙᵢ in the same ward as the infected patient 210. In another embodiment, the RPA may not include all patients with a non-zero risk score Rₙᵢ.

In operation 150, after the RPA is identified, additional operations include taking specific precautions to lower or prevent the risk of infection to the patients within the risk prone area. This may include assigning different employees, nurses, or other healthcare personnel to the not-infected patients to prevent cross-contamination with the infected patient, quarantining the infected patient, moving the infected patient, moving the not-infected patients (or at least ones having a risk score above a predetermined threshold level, and/or taking other actions to prevent infection). The actions to be taken to lower the risk score of the not-infected patients may be determined using, for example, an algorithm based on a Markov decision process or reinforcement leaning algorithm. See, e.g., https://inst.eecs.berkeley.edu/˜cs188/fa06/Handouts/mdps.pdf and https://en.wikipedia.org/wiki/ Markov decision process for examples of a Markov decision processes that may be used.

An example of taking these additional actions is illustrated in FIG. 2D, where the infected patient 210 is moved to a remote or isolated section of the ward where no or fewer patients are located. When the infected patient is moved, operations 130 and 140 may be repeated to determine new risk scores for the not-infected patients, which may results in the determination of a new risk prone area 298. In the present example, only the patient in bed 280 has a risk score that is not zero among the patients who are not infected. Even in this case, the patient in bed 280 has a very low risk of infection (e.g., as indicated by the light shading) because of the movement of the infected patient and the extra precautions taken to prevent the spread of infection.

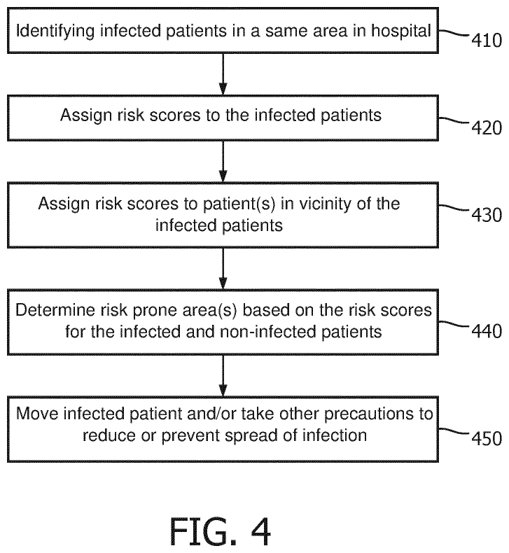

FIG. 4 illlustrates another embodiment of a method for managing the spread of infection and disease in a medical facility. In this embodiment, a patient who is not infected is between (or otherwise in the vicinity of) two or more patients who are infected. All three patients may be in the same ward, treatment area, care unit, floor, zone, room, or other location in a hospital where infection may spread, as previously indicated.

In operation 410, the method includes identifying the infected patients, for example, by name (or other identifier) and/or location in the hospital. In one embodiment, the patient may be identified by location in the hospital, e.g,. patient bed, patient room, area of patient treatment, and/or other location where patients may reside or otherwise be cared for.

In operation 420, an infection risk score Rᵢ is assigned to the infected patients identified in operation 410. The risk score Rᵢ for each of the infected patients may be determined in the same manner as operation 120, e.g., based on factors including the length of time the patient has been infected, the severity of the infection, and the course of antibiotics (or other medicine or treatment) the patient is being given. The risk scores Rᵢ may have higher values for infected patients who present a greater risk of infection to others, e.g., who are considered to have infections considered to be more highly contagious than other types of infections.

In operation 430, a risk score Rₙᵢ is assigned to patients who are not considered to be infected but who are in the vicinity of the infected patients. In the example under consideration, there is one not-infected patient between two infected patients. The risk score(s) Rₙᵢ for the patient(s) who are not infected may be calculated by an algorithm that may take the following factors into consideration: proximity of the bed of an infected patient to the beds of patients who are not infected, the type of separator between the bed of the infected patient and the beds of the not-infected patient (e.g., wall, curtain, hallway, no separator such that the beds of the infected and not-infected patients are in same room), identified pathogen of infection (e.g., highly contagious, moderately contagious, not very contagious), type of exposure (e.g., patients sharing the same nurse, cleaners, healthcare providers, or other members or employees of the hospital, patients who have undergone the same procedures or treatments, patients having comorbidity, etc.), as well as other factors that may influence separation between the bed of the infected patient and the beds of not-infected patients.

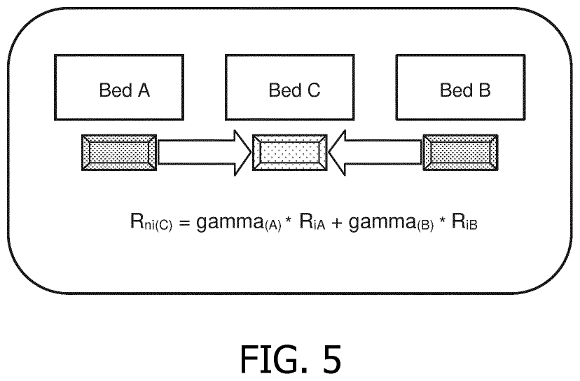

In one embodiment, the risk score for the not-infected patient may be calculated based on a sum of the values generated when Equation 1 is applied to each infected patient. For example, consider the case where a patient C who is not infected is between two patients A and B who are infected. Patients A and B may have risk scores Rᵢ that are the same or different (e.g., because they have different infections or are in different stages of the same infection). Thus, being closer to an infected patient having a higher risk score or lower risk score may change the risk score Rₙᵢ ultimately calculated for patient C. In the present case, as shown in FIG. 5, patient C is assumed to be the same distance away from infected patients A and B.

An example of an algorithm that may take this situation into consideration may calculate the risk score for patient C based on Equation 2.

R_{ni(C)}=Gamma_{(A)}*R_{iA}+Gamma_{(B)}*R_{iB} (2)

where R_{ni(C)} is the risk score for not-infected patient C that is calculated based on the sum of the risk score for patient C individually calculated relative to infected patient A and the risk score for patient C individually calculated relative to infected patient B. Because the various factors previously described in calculating Equation 1 may equally apply in this embodiment, the gamma values Gamma(a) and Gamma(B) used to calculate the risk scores relative to patients A and B may be the same or different, and the risk scores R_{iA} and R_{iB} may be the same or different based on the factors previously described.

In operation 440, at least one risk prone area RPA (or “hot spot”) is determined based on the risk scores Rᵢ and Rₙᵢ for the infected and not-infected patients. In some embodiments, determining the RPA may be optional, especially in the case where the concern is lowering the risk of infection to patient C between the two infected patients.

In operation 450, after the RPA(s) is identified, additional operations include taking specific precautions to lower or prevent the risk of infection to the patients within the risk prone area. This may include assigning different employees, nurses, or other healthcare personnel to the not-infected patients to prevent cross-contamination with the infected patient, quarantining the infected patient, moving the infected patient, moving the not-infected patients (or at least ones having a risk score above a predetermined threshold level, and/or taking other actions to prevent infection).

In one embodiment, one of two measures may be used to lower or prevent the risk of infection to the not-infected patient C between infected patients A and B. First, the patient who is not infected may be moved to another (e.g., private or semi-private) room or ward, where he may receive the same level of care. At this time, various precautions may be taken to prospectively treat the possibility of infection, or to treat the early stages of the infection if already contracted. In another case, the infected patients may be moved to another room, e.g., a private room equipped with one or more infection-prevention features.

Second, an optimization algorithm may be implemented to determine the best possible location to move the not-infected patient or the infected patients given the current circumstances in the hospital and the current level of care. Such an algorithm may be used, for example, when there is a fixed number of places an infected patient can be placed in the hospital and when it is not possible to find an isolated or private room with infection precautions in which to place the infected patients.

One example of an optimization algorithm uses a Markov decision process (MDP) as the framework (e.g., mapping states, actions, rewards) for determining the best possible location to move one or more of the infected patients. This framework helps in mapping the hospital environment as reinforcement learning problem.

The Markov decision process may be implemented by defining states and actions relative to each infected patient. The states may include or be indicative of, for example, the infected patient's bed in the current care level and moving the patient from one state (patient bed) to another. The actions may include ones taken by the hospital to change the state of the infected patient. For example, one action may correspond to moving the infected patient or performing another action that transitions the state of the infected patient, for example, in order to lower spread of the infection. In the Markov decision process, an outcome (e.g., reward) may be generated that will increase as the risk for surrounding patients decreases.

Once a set of states, actions, and rewards are defined, a Markov chain may be used to determine the a suitable (and preferably the best) action the hospital can take to reduce or prevent the risk of the infection from spreading. This may be accomplished, for example, using Bellman's equation, as indicated in Equation 3.

V(s)=maxₐ(R(s, a)+γV(s′)) (3)

In Equation 3, V(s) is the total reward produced (in a hospital scenario) in terms of reducing the threat posed by the infected patient to the surrounding area, R(s,a) is the risk the infected patient poses when the hospital takes an action a, and γ is a discounted factor for the risk posed to the surrounding patients. In one embodiment, risk and the R value are inversely proportional, e.g., the lesser the risk higher the R(s,a) value. Also, the higher the value of V(s), the less threat the infected patient poses to the surroundings. Therefore, V(s) is the sum of the risk the infection poses and the discounted value of the risk posed to beds surrounding V(s′) the infected patient. Equation 3, therefore, calculates the total risk posed by an infected patient under the states and actions used to define the Markov chain.

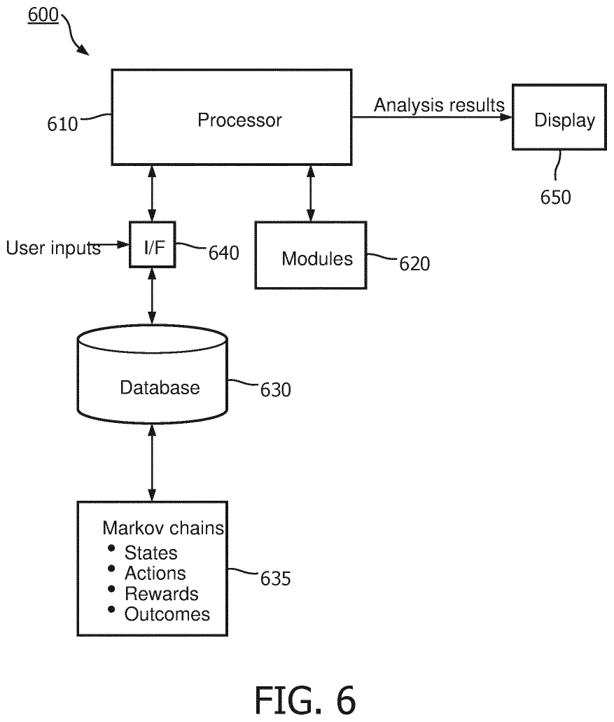

FIG. 6 illustrates an embodiment of a processing system 600 for managing the spread of infection and disease (e.g., HAIs) in a medical facility. The processing system includes a processor 610, a machine-readable storage medium 620, a database 630, an interface 640, and a display 650. The processor 610 may be implemented in logic which, for example, may include hardware, software, or both. When implemented at least partially in hardware, the processor 610 may be, for example, any one of a variety of integrated circuits including but not limited to an application-specific integrated circuit, a field-programmable gate array, a central processing unit, a combination of logic gates, a system-on-chip, a microprocessor, or another type of processing or control circuit.

When implemented in at least partially in software, the processor 610 may include or be coupled to a memory or other storage device (e.g., medium 620) for storing code or instructions to be executed, for example, by a computer, processor, microprocessor, controller, or other signal processing device. Because the algorithms that form the basis of the methods (or operations of the computer, processor, microprocessor, controller, or other signal processing device) are described in detail, the code or instructions for implementing the operations of the method embodiments may transform the computer, processor, controller, or other signal processing device into a special-purpose processor for performing the operations and methods of the embodiments described herein.

The machine-readable storage medium 620 stores instructions for controlling the processor 610 to perform some or all of the operations of the method embodiments described herein. In this case, the modules, stages, and/or other features may be implemented in any of the forms of logic (software, hardware, or a combination) herein.

The database 630 stores various forms of information that may be generated and/or used by processor 610 to perform one or more of the aforementioned operations. In one embodiment, the database 630 may store data to be used in identifying whether patients are infected or not, risk scores generated for infected patients, the risk scores generated for not-infected patients, information identifying risk prone areas (or hot spots), and protocols for managing and lowering the risk of the spread of infection given the calculated scores in the hot spot areas. The database 630 may be or include a centralized database, a decentralized database (e.g., blockchain), or a storage network of databases respectively storing the aforementioned scores and other information, for access and review by management or other personnel in a hospital network. In one embodiment, the database 630 may be at least partially implemented in a cloud-based network.

The interface 640 may be implemented in hardware, software, or both. When implemented in hardware, the interface 640 may include a port, connector, pin configuration, cable, or signal lines. In one embodiment, the interface may include a wireless interface (e.g., WiFi, GSM, CDMA, LTE, or other mobile network), or an interface compatible with another type of communication protocol). The interface 640 may transfer information between the processor 610 and the database 630, including but not limited to data generated based on operations of the modules 620. The interface 640 may also receive information from a user to control the processor and modules, e.g., to update the processor or modules with new, different, or updated parameters, etc.

In one case, the processor 610 may be located remotely from the display 650, e.g., may be included in a virtual private network accessible by personnel at different locations. When implemented in software, an interface between the processor 610 and display 650 may include, for example, application programming interface (API) running on a workstation, server, client, or mobile device.

In operation, the instructions stored in the machine-readable medium 620 controls the processor 610 to perform the operations of the method and system embodiments described herein, including implementing the algorithms, Markov decision processes, equations, and other aspects of the disclosed embodiments. The processor may receive inputs from one or more users, applications, and/or control software during this time to control, change, or implement some of these operations. The results of the processor 610, including risk scores, identification of hot spot areas, generated outcomes and probabilities, etc., may be displayed on the display 650.

Technical Innovation

The ability to accurately detect health risks posed by the spread of infectious disease in a hospital or other medical facility is of paramount importance, not only for employees, care professionals, and patients in these facilities but also to prevent the occurrence of an epidemic. For persons with underdeveloped or compromised immune systems, preventing infections may be a life or death matter. Presently, there are no reliable ways for determining areas in medical facilities that are harboring pathogens or which otherwise may be labeled as “hot spots” for exposure to infection.

In accordance with one or more embodiments, a system and method are provided which identify areas in a medical facility that are prone to the spread of infection. Probabilistic outcomes are then generated for reducing or preventing the risk to patients in those areas. In one embodiment, different risk scenarios are contemplated and algorithms are used to generate and assign scores to patients in the entire facility or in selected areas of the facility. The scores are then used as a basis for identifying the greatest threats in a given area. Through an analysis (which may or may not be performed using a Markov chain), outcomes may then be generated to optimize actions for guiding healthcare professionals in isolating infected patients or protecting patients who have not yet been infected. These actions may include, for example, moving infected or not-infected patients to various locations that produces the lowest risk of infection. The actions may include, for example, moving patients to protected or isolated rooms, rearranging the locations of patients in a ward in a optimal way, enforcing protocols (e.g., sanitizing procedures, etc.) to reduce risk scores, and instructing hospital staff to complete various tasks before entering an area identified as a hot spot for the spread of infection.

While one or more features of the embodiments may involve the use of a mathematical formula, the embodiments are in no way restricted solely to a mathematical formula. Nor are they directed to a method of organizing human activity or a mental process. Rather, the complex and specific approach taken by the embodiments, combined with the amount of information processing performed, negate the possibility of the embodiments being performed by human activity or a mental process. Moreover, while a computer or other form of processor may be used to implement one or more features of the embodiments, the embodiments are not solely directed to using a computer as a tool to otherwise perform a process that was previously performed manually.

Nor do these embodiments preempt the general concept of making healthcare cost decisions. Rather, the embodiments disclosed herein take a specific approach (e.g., through event logs, trace sets, clustering algorithms, and weighting and distance measuring models) to solving technological problems that do not preempt, or otherwise restrict the public from practicing the general concept of, allocating healthcare resources.

The methods, processes, and/or operations described herein may be performed by code or instructions to be executed by a computer, processor, controller, or other signal processing device. The code or instructions may be stored in a non-transitory computer-readable medium in accordance with one or more embodiments. Because the algorithms that form the basis of the methods (or operations of the computer, processor, controller, or other signal processing device) are described in detail, the code or instructions for implementing the operations of the method embodiments may transform the computer, processor, controller, or other signal processing device into a special-purpose processor for performing the methods herein.

The modules, stages, models, processors, and other information generating, processing, and calculating features of the embodiments disclosed herein may be implemented in logic which, for example, may include hardware, software, or both. When implemented at least partially in hardware, the modules, models, engines, processors, and other information generating, processing, or calculating features may be, for example, any one of a variety of integrated circuits including but not limited to an application-specific integrated circuit, a field-programmable gate array, a combination of logic gates, a system-on-chip, a microprocessor, or another type of processing or control circuit.

When implemented in at least partially in software, the modules, models, engines, processors, and other information generating, processing, or calculating features may include, for example, a memory or other storage device for storing code or instructions to be executed, for example, by a computer, processor, microprocessor, controller, or other signal processing device. Because the algorithms that form the basis of the methods (or operations of the computer, processor, microprocessor, controller, or other signal processing device) are described in detail, the code or instructions for implementing the operations of the method embodiments may transform the computer, processor, controller, or other signal processing device into a special-purpose processor for performing the methods herein.

It should be apparent from the foregoing description that various exemplary embodiments of the invention may be implemented in hardware. Furthermore, various exemplary embodiments may be implemented as instructions stored on a non-transitory machine-readable storage medium, such as a volatile or non-volatile memory, which may be read and executed by at least one processor to perform the operations described in detail herein. A non-transitory machine-readable storage medium may include any mechanism for storing information in a form readable by a machine, such as a personal or laptop computer, a server, or other computing device. Thus, a non-transitory machine-readable storage medium may include read-only memory (ROM), random-access memory (RAM), magnetic disk storage media, optical storage media, flash-memory devices, and similar storage media and excludes transitory signals.

It should be appreciated by those skilled in the art that any blocks and block diagrams herein represent conceptual views of illustrative circuitry embodying the principles of the invention. Implementation of particular blocks can vary while they can be implemented in the hardware or software domain without limiting the scope of the invention. Similarly, it will be appreciated that any flow charts, flow diagrams, state transition diagrams, pseudo code, and the like represent various processes which may be substantially represented in machine readable media and so executed by a computer or processor, whether or not such computer or processor is explicitly shown.

Accordingly, it is to be understood that the above description is intended to be illustrative and not restrictive. Many embodiments and applications other than the examples provided would be apparent upon reading the above description. The scope should be determined, not with reference to the above description or Abstract below, but should instead be determined with reference to the appended claims, along with the full scope of equivalents to which such claims are entitled. It is anticipated and intended that future developments will occur in the technologies discussed herein, and that the disclosed systems and methods will be incorporated into such future embodiments. In sum, it should be understood that the application is capable of modification and variation.

The benefits, advantages, solutions to problems, and any element(s) that may cause any benefit, advantage, or solution to occur or become more pronounced are not to be construed as a critical, required, or essential features or elements of any or all the claims. The invention is defined solely by the appended claims including any amendments made during the pendency of this application and all equivalents of those claims as issued.

All terms used in the claims are intended to be given their broadest reasonable constructions and their ordinary meanings as understood by those knowledgeable in the technologies described herein unless an explicit indication to the contrary in made herein. In particular, use of the singular articles such as “a,” “the,” “said,” etc. should be read to recite one or more of the indicated elements unless a claim recites an explicit limitation to the contrary.

The Abstract of the Disclosure is provided to allow the reader to quickly ascertain the nature of the technical disclosure. It is submitted with the understanding that it will not be used to interpret or limit the scope or meaning of the claims. In addition, in the foregoing Detailed Description, it can be seen that various features are grouped together in various embodiments for the purpose of streamlining the disclosure. This method of disclosure is not to be interpreted as reflecting an intention that the claimed embodiments require more features than are expressly recited in each claim. Rather, as the following claims reflect, inventive subject matter lies in less than all features of a single disclosed embodiment. Thus the following claims are hereby incorporated into the Detailed Description, with each claim standing on its own as a separately claimed subject matter.

We claim:

1. A method for processing medical information to determine an infection risk prone area of a medical facility, comprising:
identifying a first patient in a first state;
identifying a second patient in a second state;
calculating a first risk score for the first patient;
calculating a first risk score for the second patient; and
determining a risk prone area in the medical facility based on the first risk score for the first patient and the first risk score for the second patient, wherein the first state is an infected state and the second state is different from the first state and wherein the first risk score of the first patient provides an indication of a severity of the infected state of the first patient and the first risk score of the second patient provides an indication of the second patient being infected by the first patient.

2. The method of claim 1, wherein the first risk score of the second patient is calculated based on the first risk score of the first patient and a gamma value, the gamma value corresponding to a probability that the second patient will be infected by the first patient.

3. The method of claim 2, further comprising:
calculating the gamma value based on a location of the second patient relative to a location of the first patient in the medical facility.

4. The method of claim 2, further comprising:
calculating the gamma value based on a type of separator between the first patient and the second patient.

5. The method of claim 2, further comprising:
calculating the gamma value based on one or more procedures or protocols in place at the medical facility.

6. The method of claim 1, further comprising:
determining a first location in the medical facility to move the second patient relative to a location of the first patient,
calculating a second risk score for the second patient at the first location, and
selecting the first location if the second risk score for the second patient indicates a lower probability that the second patient will be infected by the first patient than the first risk score of the first patient.

7. The method of claim 1, further comprising:
identifying one or more actions to reduce the first risk score of the second patient.

8. The method of claim 1, further comprising:
identifying a third patient in the first state;
calculating a risk score for the third patient;
calculating a second risk score for the second patient based on the risk score of the third patient; and
calculating a third risk score for the second patient based on the first risk score of the second patient and the third risk score for the second patient.

9. The method of claim 8, wherein the first patient and the third patient have different infections or are in different stages of a same infection.

10. The method of claim 8, wherein the risk score of the third patient is different from the first risk score of the first patient.

11. The method of claim 8, further comprising:
determining a plurality of locations in the medical facility to move the second patient relative to locations of the first and third patients; and
selecting one of the plurality of locations using a Markov chain that generates different probabilities corresponding to the plurality of locations.

12. A system for processing medical information, comprising:
a storage area to store an algorithm;
a processor configured to implement the algorithm to:
a) calculate a first risk score for a first patient in a first state;
b) calculate a first risk score for a second patient in a second state; and
c) determine a risk prone area in a medical facility based on the first risk score for the first patient and the first risk score for the second patient, wherein the first state is an infected state and the second state is different from the first state and wherein the first risk score of the first patient provides an indication of a severity of the infected state of the first patient and the first risk score of the second patient provides an indication of the second patient being infected by the first patient.

13. The system of claim 12, wherein the processor is configured to:
calculate the first risk score of the second patient based on the first risk score of the first patient and a gamma value, the gamma value corresponding to a probability that the second patient will be infected by the first patient.

14. The system of claim 13, wherein the processor is configured to:
calculate the gamma value based on at least one of a location of the second patient relative to a location of the first patient in the medical facility, a type of separator between the first patient and the second patient, or one or more procedures or protocols in place at the medical facility.

15. The system of claim 12, wherein the processor is configured to:
determine a first location in the medical facility to move the second patient relative to a location of the first patient,
calculate a second risk score for the second patient at the first location, and
select the first location if the second risk score for the second patient indicates a lower probability that the second patient will be infected by the first patient than the first risk score of the first patient.

16. The system of claim 12, wherein the processor is configured to identify one or more actions to reduce the first risk score of the second patient.

17. The system of claim 12, wherein the processor is configured to:
identify a third patient in the first state;
calculate a risk score for the third patient;
calculate a second risk score for the second patient based on the risk score of the third patient; and
calculate a third risk score for the second patient based on the first risk score of the second patient and the third risk score for the second patient.

18. The system of claim 17, wherein the processor is configured to:
determine a plurality of locations in the medical facility to move the second patient relative to locations of the first and third patients; and
select one of the plurality of locations using a Markov chain that generates different probabilities corresponding to the plurality of locations.

19. A non-transitory, machine-readable medium storing instructions for controlling a processor to perform operations which include:
calculating a first risk score for a first patient in a first state;
calculating a first risk score for a second patient in a second state; and
determining a risk prone area in a medical facility based on the first risk score for the first patient and the first risk score for the second patient, wherein the first state is an infected state and the second state is different from the first state and wherein the first risk score of the first patient provides an indication of a severity of the infected state of the first patient and the first risk score of the second patient provides an indication of the second patient being infected by the first patient.

20. The medium of claim 19, wherein the instructions are to control the processor to:
calculate the first risk score of the second patient based on the first risk score of the first patient and a gamma value, the gamma value corresponding to a probability that the second patient will be infected by the first patient.
